(19) United States
(12) Plant Patent Application Publication
(10) Pub. No.: US 2004/0210975 P1
(43) Pub. Date: Oct. 21, 2004

(54) Osteospermum plant named of 'Sunny Henry'

(76) Inventor: Bjarne N. Larsen, Odense N (DK)

Correspondence Address: FOLEY AND LARDNER, SUITE 500, 3000 K STREET NW, WASHINGTON, DC, 20007, US

(21) Appl. No.: 10/788,298

(22) Filed: Mar. 1, 2004

(30) Foreign Application Priority Data
Jul. 3, 2002 (QZ) 2002/0996

(51) Int. Cl. A01H 5/00

(52) U.S. Cl. PLT/360

(57) ABSTRACT

A new distinct cultivar of Osteospermum plant named ‘Sunny Henry’, characterized by white flower color RHS 158D with slight yellow tinge, to pure white, RHS 155D; dense and bushy plant form, mainly due to more upright stems; vigorous growth habit; less need for chemical growth retardation; and higher number of flowers per plant.

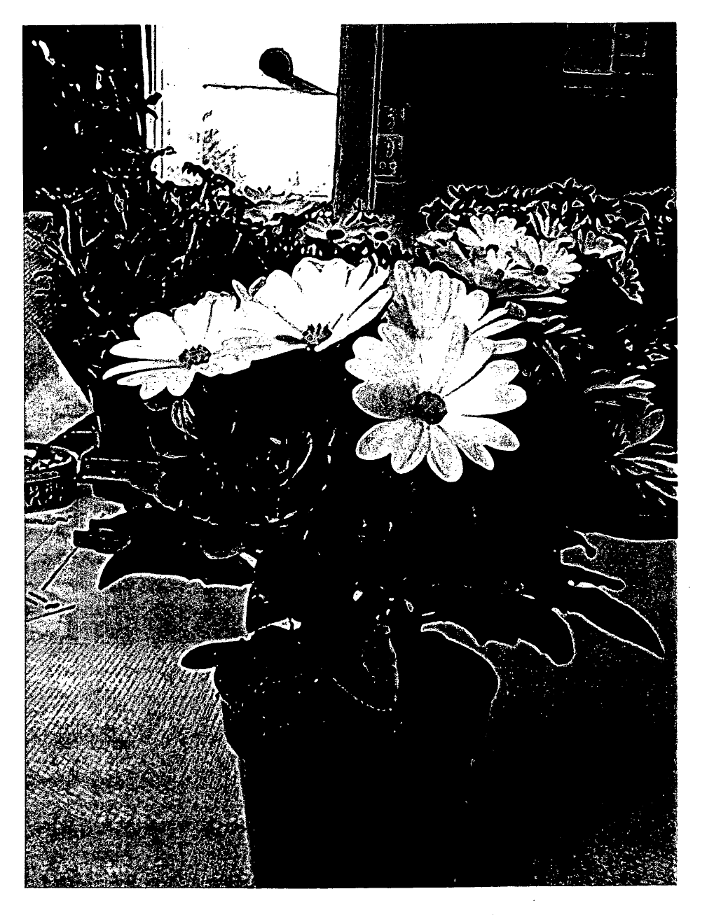

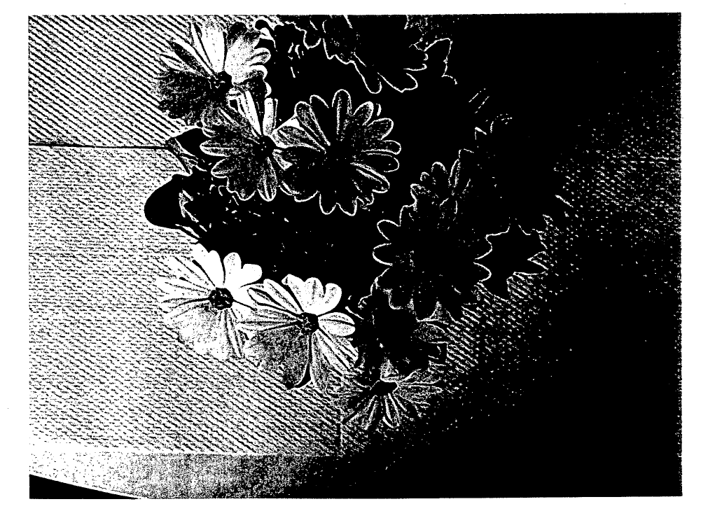

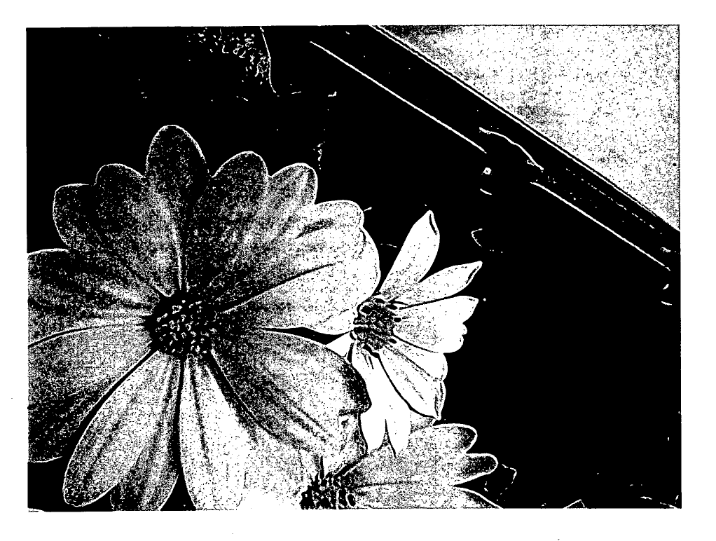

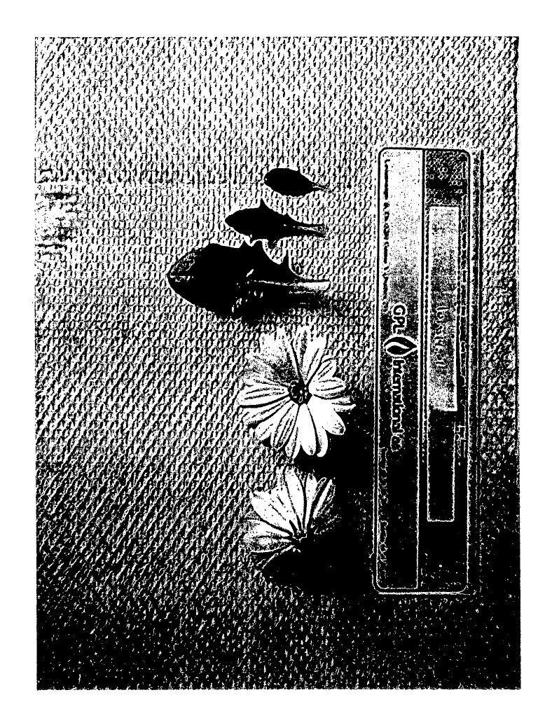

### GENUS AND SPECIES OF THE PLANT CLAIMED

Osteospermum eclonis (DC) T Norl.

### VARIETY DENOMINATION

Sunny Henry

### BACKGROUND OF THE INVENTION

The present Invention relates to a new and distinct cultivar of Osteospermum plant, botanically known as Osteospermum eclonis (DC) T Norl., commonly known by the name Cape Daisy, and hereinafter referred to by the name ‘Sunny Henry’.

The new Osteospermum is a product of a planned breeding program conducted by the Inventor, Bjarne N. Larsen, in Stige, Denmark. The Inventor selected the new Osteospermum cultivar on the basis of its flower color and compact, freely branching habit. Plants of the new Osteospermum are more upright, compact and have a unique color combination of upper and underside of the ray petals.

Asexual reproduction of the new cultivar by terminal vegetative cuttings taken and propagated during trial production batches in Stige, Denmark, has shown that the unique features of this new Osteospermum are stable and reproduced true to type in many successive generations.

### BRIEF SUMMARY OF THE INVENTION

Plants of the cultivar ‘Sunny Henry’ have not been observed under all possible environmental conditions. The phenotype may vary somewhat with variations in environment such as temperature, light intensity, day length, and fertility level without, however, any variance in genotype.

The following traits have been repeatedly observed and are determined to be the unique characteristics of ‘Sunny Henry’. The following characteristics in combination distinguish ‘Sunny Henry’ as a new and distinct cultivar:

1. White flower color RHS 158D with slight yellow tinge, to pure white, RHS 155D.

2. Dense and bushy plant form, mainly due to more upright stems.

3. Vigorous growth habit; less need for chemical growth retardation.

4. Higher number of flowers per plant.

Plants of the cultivar ‘Sunny Henry’ can be compared to plants of the Osteospermum eclonis (DC) T Norl. cultivar ‘Sunny Gustav’ (U.S. Plant Pat. No. 10,670). In side-by-side comparisons conducted by the Inventor in Stige Denmark, plants of the cultivar ‘Sunny Henry’ and the cultivar ‘Sunny Gustav’ differ in the following characteristics:

1. ‘Sunny Henry’ has striking white ray flowers and yellow disk with orange pollen, while ‘Sunny Gustav’ has a white flower with blue eye.

2. ‘Sunny Henry’ has yellow-green-colored leaves while plants of the cultivar ‘Sunny Gustav’ have darker green leaves.

3. ‘Sunny Henry’ has shorter peduncles than plants of the cultivar ‘Sunny Gustav’.

4. ‘Sunny Henry’ is shorter and more compact than the plants of the cultivar ‘Sunny Gustav’.

5. ‘Sunny Henry’ has more and larger flowers per plant than the plants of the cultivar ‘Sunny Gustav’.

### BRIEF DESCRIPTION OF THE PHOTOGRAPHS

The accompanying color photographs illustrate the overall appearance and details of flower form color and structures of the new cultivar, showing the colors as true as it is reasonably possible to obtain in color reproductions of this type. Colors in the photographs may differ slightly from the color values cited in the detailed botanical description, which more accurately describe the actual colors of the new Osteospermum.

The first photograph comprises a side perspective view of a typical flowering plant of ‘Sunny Henry’ as grown in an 11 cm pot. The second photo is a close-up of the composite young and older flowers of ‘Sunny Henry’. The third photograph is a top view of a typical flowering plant of ‘Sunny Henry’. The fourth photograph shows the flowers and leaves of ‘Sunny Henry’ (in the fourth photograph, ‘Sunny Henry’ is labelled by its breeder's reference no. ‘10.514.01’).

### DETAILED BOTANICAL DESCRIPTION

In the following description, color references are made to The Royal Horticultural Society Colour Chart, 4^{th} edition, where general terms of ordinary dictionary significance are used. Plants were grown under greenhouse conditions. The plants described were grown for about 20 weeks after cutting.

Plant description:

Form.—Perennial plant with upright, globular plant habit; composite flower heads; freely branching with lateral flowering branches forming at every node; dense and bushy.

Crop time.—After rooting, about 18 weeks are required to produce finished flowering plants in 11 cm pots.

Plant height (soil level to top of plant plane).—About 15 cm.

Plant width.—18 cm.

Vigor.—Vigorous growth rate.

Time to initiate roots.—About 10 to 14 days at 18 to 21 C in tunnels in a greenhouse.

Root description.—Fine, well branched

Foliage description.—Leaves alternate, single, dentate, brochidodromus venation. Length: 3-9 cm. Width: About 30 mm. Shape: Obovate to lanceolate. Apex: obtuse. Base: attenuate. Margin: broadly dentate. Texture: smooth, glabrous, shiny. Scattered short, stiff hairs. Color: Young foliage, upper and lower surfaces: 147B, yellow-green. Mature foliage upper surfaces N147A and and lower surface 148A Venation, 148B.

Flower description:

Flower arrangement and shape.—Tubular disk and petalled ray flowers in composite daisy heads; with 15 parted involucre.

Natural flowering season.—Continuous throughout the spring and summer. Season can be extended by vernalization and long day treatments. Hot weather above 30 C reduces flowering.

Flower longevity on the plant.—Longevity of individual flowers is highly dependent on temperature and light conditions 5 to 9 days. Flowers persistent, petals folding and withering slowly.

Inflorescence size.—Diameter about 6 cm.

Flowers.—Length: Ray petals: About 27 mm. Width: About 8 mm. Lanceolate, acute ray petal lobes. Ray petal color: Upper surfaces, 158D with slight yellow tinge under sides: 1B edges 1D to 1A at maturity. Disk flowers N155D, white.

Peduncle.—Strength: Moderately strong. Length: About 6 cm. Diameter: About 3 mm. Color: 144C yellow-green.

Reproductive organs:

Anthers.—Linear, fused, stamen color 17A, yellow.

Pollen.—Color 17A yellow.

Pistil and stigma.—Color 16C yellow-orange.

Seed.—Length: About 1 mm. Diameter: About 0.3 mm.

Weather tolerance.—Plants of the new Osteospermum have exhibited good tolerance to draught, rain and wind, and low temperatures to −1 C.

I claim:

1. A new and distinct cultivar of Osteospermum plant named ‘Sunny Henry’, as illustrated and described herein.
